(19) United States
(12) Patent Application Publication
(10) Pub. No.: US 2010/0016363 A1
(43) Pub. Date: Jan. 21, 2010

(54) Fixed Combination Dosage Forms for the Treatment of Migraine

(75) Inventors: William R. Maichle, Lowell, NC (US); Carl L. Whatley, Montgomery, AL (US); Giorgio Reiner, Como (IT); Alberto Reiner, Como (IT)

Correspondence Address: PATENT CORRESPONDENCE;ARNALL GOLDEN GREGORY LLP, 171 17TH STREET NW, SUITE 2100, ATLANTA, GA, 30363, US

(73) Assignees: Kowa Pharmaceuticals America, Inc., Montgomery, AL (US); APR Applied Pharma Research, S.A., Balerna (CH)

(21) Appl. No.: 12/298,550

(22) PCT Filed: Apr. 25, 2007
(86) PCT No.: PCT/US07/09953

§ 371 Date: Apr. 1, 2009

Related U.S. Application Data
(60) Provisional application No. 60/795,214, filed on Apr. 25, 2006.

(51) Int. Cl. A61K 31/454; A61K 31/196; A61K 31/4045; A61K 31/4196; A61K 31/422; A61K 31/405; A61P 25/22

(52) U.S. Cl. 514/323; 514/567; 514/415; 514/383; 514/376

(57) ABSTRACT

Therapeutic regimens and dosage forms are disclosed for the treatment of migraine headache. The regimens preferably combine a serotonin receptor agonist, such as sumatriptan, eletriptan or almotriptan, with a fast acting formulation of diclofenac potassium.

### RELATED APPLICATIONS

The present application claims priority under 35 U.S.C. § 119 to U.S. Ser. No. 60/795,214, filed Apr. 25, 2006.

### FIELD OF THE INVENTION

The present invention relates to therapeutic regimens and dosage forms for the treatment of migraine and accompanying symptoms. The regimens preferably combine a known serotonin receptor agonist of the triptan family with a short acting non-steroidal anti-inflammatory drug (“NSAID”), such as diclofenac potassium.

### BACKGROUND OF THE INVENTION

Diclofenac is a non-steroidal anti-inflammatory drug that is widely prescribed for inflammatory conditions such as osteoarthritis and rheumatoid arthritis. Diclofenac inhibits the enzymes cyclooxygenase-1 and cyclooxygenase-2, which in turn mediate prostaglandin synthesis. Diclofenac is widely prescribed for the treatment of acute pain and is used in a number of countries to treat migraine attacks. Migraines are recurrent, often familial, symptom complexes of periodic attacks of vascular headache, that affect approximately 17% of adult women and 6% of adult men. Stewart et al., NEUROLOGY, 1994, 44 (suppl. 4), 517-523.

Sumatriptan is a serotonin receptor agonist that causes constriction of cranial blood vessels. Sumatriptan is widely prescribed in the United States and around the world as a treatment for acute migraine headaches with our without aura in adults. Sumatriptan succinate is marketed commercially as Imitrex® in tablet, nasal spray and injectable dosage forms. Doenicke et al., Lancet, 1988, Vol. 1, 1309-11; and Feniuk & Humphrey, Drug Development Research, 1992, 26, 235-40. It is the prototypical example of a class of compounds that have recently been classified as 5-HT_{1B/1D} receptor agonists. Hartig et al., TIPS, 1996, 17, 103-105.) Other marketed drugs that are classified as 5-HT_{1B/1D} receptor agonists include rizatriptan, eletriptan, zolmitriptan, naratriptan, almotriptan, and frovatriptan.

Sumatriptan is reported to suffer from several disadvantages, including a particularly unpleasant taste which, when administered orally, may exacerbate the nausea and vomiting often associated with migraine. In an effort to solve this problem, Phillips et al., in U.S. Pat. No. 5,863,559, propose formulating the drug in a film coated tablet.

Sumatriptan also suffers from reports of migraine reoccurrence. In particular, it has been observed that the migraine in patients who are treated with sumatriptan often reoccurs within 8 or 24 hours of an initial treatment. To overcome this problem, Plachetka et al., in U.S. Pat. No. 6,060,499, have proposed combining the sumatriptan with a long acting NSAID such as naproxen sodium. According to Plachetka, “the addition of a long-acting NSAID to a 5-HT agonist extends the period of effective anti-migraine action and prevents the relapse headache from occurring (or “rebound migraines”), whatever is its cause.” See also Smith et al., HEADACHE 2005; 45:983-91; and U.S. Pat. No. 6,384,034 to Simitchieva et al. (proposing a combination of sumatriptan or rizatriptan and a selective COX-2 inhibitor such as rofecoxib or celecoxib.))

### SUMMARY OF THE INVENTION

Contrary to the teachings of the prior art, which recommend that serotonin agonists such as the triptans be combined with a lone acting NSAID to minimize the risk of rebound migraine, the current invention combines a serotonin agonist with a short acting NSAID that has preferably been specially formulated to deliver the NSAID rapidly over a short period of time. The combination will work to significantly decrease the incidence of rebound migraine even with sumatriptan—a drug often criticized due to its short half-life. Preferred serotonin agonists for practicing the current invention include sumatriptan, eletriptan, zolmitriptan, naratriptan, almotriptan, frovatriptan and rizatriptan.

The preferred NSAID is diclofenac potassium, and it is preferably specially formulated in a rapidly bioavailable solid oral dosage form that attains a high Cₘₐₓ in the bloodstream in less than about twenty minutes.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

The accompanying drawing, which is incorporated in and constitutes a part of this specification, illustrates several embodiments of the invention and together with the description, serves to explain the principles of the invention.

FIG. 1 contains a graphical summary of headache intensities during the first twenty four hours after treatment, comparing a fast release diclofenac sachet formulation and placebo, as described in Example 3.

### DESCRIPTION OF THE INVENTION

The present invention may be understood more readily by reference to the following detailed description of preferred embodiments of the invention and the Examples included therein.

### DEFINITIONS

As used in the specification and claims, the singular forms a, an and the include plural references unless the context clearly dictates otherwise. For example, the term a pharmaceutical excipient may refer to one or more pharmaceutical excipients for use in the presently disclosed formulations and methods.

USP means the United States Pharmacopeia and National Formulary (USP 28-NF 23) Rockville, Md.: United States Pharmacopeia Convention; 2004, unless stated to the contrary. USP 28 <701> refers to physical test 701, disintegration, contained on pages 2411-2412 of the USP. USP 28 <711> refers to physical test 711, dissolution, contained on pages 2412-2414 of the USP. Measurements are made in phosphate buffered water at pH=6.8 and 37° C. (diclofenac), or in 0.01M hydrochloric acid buffer at 37° C. (sumatriptan), or in deaerated water at pH 7.0 and 37° C. (rizatriptan), or in 0.1M hydrochloric acid buffer at 37° C. (other actives), and measured in a Type II dissolution apparatus at 50 rpm, according to USP 28 <711>. A preferred test for any drug is in water buffered by 0.1M HCl at 37° C. at 50 RPM using a USP Type II dissolution apparatus.

A dosage form, as used herein, refers to a formulation that is ready for administration to a subject. As used herein, it may refer to solid dosage forms, including, but not limited to, tablets, powders and capsules, tablets being the most preferred. Alternatively, it may refer to a liquid dosage form such as a solution or a suspension. An “intact” dosage form refers to a dosage form which is ingested in the form it is provided. In preferred embodiments of this invention, the dosage form is a tablet, and the tablets are ingested in an intact form.

When doses are given for a drug and its salt, it will be understood that the calculated dose is based on the molecular weight of the active pharmaceutical ingredient, which includes the cationic and anionic species in the case of a salt, and just the base when the active principle is not present as a salt.

When ranges are given by specifying the lower end of a range separately from the upper end of the range, it will be understood that the range can be defined by selectively combining any one of the lower end variables with any one of the upper end variables that is mathematically possible.

When used herein the term “about” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent to the recited strength.

### Discussion

In a first principal embodiment, the invention provides a fixed combination oral dosage form comprising a rapidly bioavailable form of a short acting NSAID, such as diclofenac or one of its pharmaceutically acceptable salts, and a 5-HT_{1B/1D} receptor agonist such as sumatriptan and eletriptan, or a pharmaceutically acceptable salt or ester thereof. The dosage form is preferably formulated specially for the treatment of migraine, and in a second principal embodiment the invention provides a method of treating one or more migraine symptoms in a patient suffering from migraine comprising concomitantly orally administering a rapidly bioavailable oral dosage form of a short acting NSAID such as diclofenac, or a pharmaceutically acceptable salt thereof, and a 5-HT_{1B/1D} receptor agonist such as sumatriptan and eletriptan, or a pharmaceutically acceptable salt or ester thereof.

A short acting NSAID is defined herein to mean an NSAID having a half life of less than about 6 hours, 5 hours, 4 hours, or even 3 or 2 hours. Diclofenac is a preferred short acting NSAID for purposes of this invention, and is chemically described as [(2,6-dichloro-anilino)-2-phenyl]-2-acetic acid. The potassium salt of the molecule is represented by the following chemical structure:

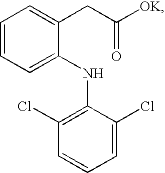

For purposes of this invention, diclofenac can be administered as the acid form or as any pharmaceutically acceptable salt that demonstrates adequate stability upon storage and bioavailability upon administration, but is preferably administered as diclofenac sodium or diclofenac potassium.

The dosage form preferably comprises from about 10 mg. to about 100 mg., more preferably from about 15 mg. to about 75 mg., from about 40 to about 60 mg., or about 25 mg. or about 50 mg. specifically, of diclofenac or diclofenac salt (as diclofenac potassium, diclofenac sodium or diclofenac acid). In addition, the dosage form preferably meets one or more of the following pharmacokinetic criteria for the diclofenac or other short acting NSAID:
a tₘₐₓ of from about 5 or 10 to about 40, 35, 30, 25 or 20 minutes, most preferably from about 10 to about 20 minutes (preferably when tested in a fasted state);
an inter-subject coefficient of variability for said tₘₐₓ of preferably less than about 80, 75, 60, 50, 45, 40, 35, 30% or 25%;
a Cₘₐₓ of from about 1200, 1300, 1400, 1500 or 1600 to about 2500 ng/ml for a 50 mg. dose of diclofenac potassium or diclofenac (i.e. 0.026 ml⁻¹ to about 0.05 ml⁻¹ when normalized), preferably from about 1300 to about 2500 ng/ml for a 50 mg. dose (i.e. from about 0.026 liter⁻¹ to about 0.05 liter⁻¹ when normalized) and more preferably from about 1500 to about 2500 ng/ml for a 50 mg. dose (i.e. from about 0.03 liter⁻¹ to about 0.05 liter⁻¹ when normalized) (preferably when tested in a fasted state);
an inter-subject coefficient of variability for said Cₘₐₓ of less than about 70, 60, 50, 45 or 40%;
a single plasma concentration peak reflecting predominant absorption in the upper portion of the gastrointestinal tract; and/or
a disintegration or dissolution time of less than about 20, 15, 10, 5 or 3 minutes when tested according to USP 28 <701> or USP 28 <711> (Q=85%).

The dosage form also preferably comprises a 5-HT_{1B/1D} receptor agonist, which is preferably selected from rizatriptan, eletriptan, zolmitriptan, naratriptan, almotriptan, and frovatriptan, in a therapeutically effective amount. Alternatively, the fast acting NSAID may be combined with another migraine agent such as dihydroergotamine or metoclopramide, also in a therapeutically effective amount. These non-NSAID ingredients may be mixed intimately with the diclofenac so that they are released from the dosage form at approximately the same rate, or they may be specially formulated for release distinct from the NSAID, as in a bilayer tablet (as discussed below). Alternatively, the non-NSAID may be coated with a suitable protective agent such as a methacrylic copolymer, for protection from the alkaline effects of the bicarbonate buffer.

Sumatriptan is chemically designated as 3-[2-(dimethylamino)ethyl]-N-methyl-indole-5-methanesulfonamide. The succinic acid salt of sumatriptan is represented by the following chemical structure:

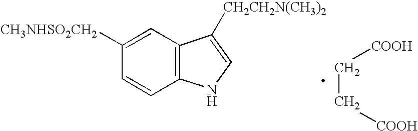

For purposes of this invention, sumatriptan can be administered as any pharmaceutically acceptable salt or ester that demonstrates adequate stability upon storage and bioavailability upon administration, but a preferred form of sumatriptan for purposes of this invention is sumatriptan succinate (1:1).

The dosage form preferably comprises from about 15 mg. to about 125 mg. of sumatriptan (based on the weight of the base, in whatever form the sumatriptan is present), and more preferably comprises from about 25 mg. to about 100 mg. of sumatriptan, or about 25 mg., 50 mg. or 100 mg. specifically, of sumatriptan (corresponding to 35, 70 or 140 mg. of sumatriptan succinate). The mean maximum concentration following oral dosing with 25 mg. is preferably about 18 ng/mL. (with a preferred range of from about 7 to about 47 ng/mL), and 51 ng/mL (range, 28 to 100 ng/mL) following oral dosing with 100 mg. of sumatriptan. In addition, the dosage form preferably yields a tₘₐₓ for the sumatriptan of from about 1.5 to about 3.0 hours, preferably from about 2.0 to about 2.5 hours, whether determined during a migraine-free period or during an attack.

Eletriptan is chemically designated as (R)-3-[(1-Methyl-2-pyrrolidinyl)methyl]-5-[2-(phenylsulfonyl)ethyl]-1H-indole, and the hydrobromide salt is represented by the following chemical structure:

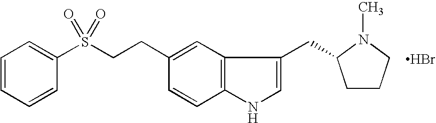

For purposes of this invention, eletriptan can be administered as any pharmaceutically acceptable salt that demonstrates adequate stability upon storage and bioavailability upon administration, but a preferred form of eletriptan for purposes of this invention is eletriptan monohydrobromide.

The dosage form preferably comprises from about 10 mg. to about 100 mg. of eletriptan (based on the weight of the base, in whatever form the eletriptan is present), and more preferably comprises from about 10 mg. to about 60 mg. of eletriptan, from about 20 to about 40 mg. of eletriptan, or about 20 mg. or about 40 mg. specifically, of eletriptan (corresponding to 24.2 mg. or 48.5 mg. of eletriptan hydrobromide). In addition, the dosage form preferably yields a tₘₐₓ for the eletriptan of from about 1.0 to about 3.0 hours, preferably about 1.5 to about 2.0 hours, whether determined during a migraine-free period or during an attack.

Rizatriptan is chemically described as N,N-dimethyl-5-(1H-1,2,4-triazol-1-ylmethyl)-1H-indole-3-ethanamine. The benzoic acid salt of rizatriptan is depicted by the following chemical structure:

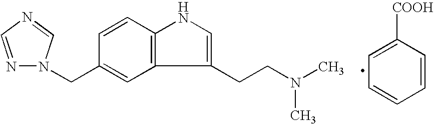

For purposes of this invention, rizatriptan can be administered as the base or as any pharmaceutically acceptable salt or ester that demonstrates adequate stability upon storage and bioavailability upon administration, but a preferred form of rizatriptan for purposes of this invention is rizatriptan benzoate.

The dosage form preferably comprises from about 2.5 mg. to about 15 mg. of rizatriptan (based on the weight of the base, in whatever form the rizatriptan is present), and more preferably comprises from about 5 mg. to about 10 mg. of rizatriptan, or about 5 mg. or about 10 mg. specifically, of rizatriptan (corresponding to 7.265 or 14.53 mg. of rizatriptan benzoate). In addition, the dosage form preferably yields a tₘₐₓ for the rizatriptan of from about 0.5 to about 3.0 hours, preferably from about 1.0 to about 2.5 hours, whether determined during a migraine-free period or during an attack.

Other 5-HT_{1B/1D} receptor agonists with which the invention could be practiced include:
Zolmitriptan
Naratriptan
Almotriptan
Frovatriptan
   Other migraine products that could be combined with the diclofenac potassium in the dosage forms of the present invention include dihydroergotamine and metoclopramide.

Zolmitriptan is chemically designated as (S)-(4)-[[3-[2-(dimethylamino)ethyl]-1H-indol-5-yl]methyl]-2-oxazolidinone, and has the following chemical structure:

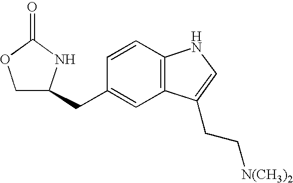

The dosage form preferably comprises from about 1.5 mg. to about 7.5 mg. of zolmitriptan, and more preferably comprises from about 2.5 mg. to about 5.0 mg. of zolmitriptan, or about 2.5 mg. or about 5.0 mg. specifically, of zolmitriptan. In addition, the dosage form preferably yields a tₘₐₓ for the zolmitriptan of from about 1.0 to about 4.0 hours, preferably from about 1.0 to about 2.0 hours, or from about 2.5 to about 3.5 hours, whether determined during a migraine-free period or during an attack.

Naratriptan is chemically designated as N-methyl-3-(1-methyl-4-piperidinyl)-1H-indole-5-ethanesulfonamide, and has the following chemical structure when present as the hydrochloride:

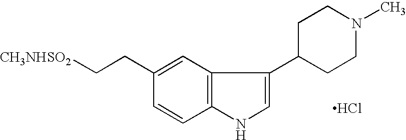

For purposes of this invention, naratriptan can be administered as the base or as any pharmaceutically acceptable salt that demonstrates adequate stability upon storage and bioavailability upon administration, but a preferred form of naratriptan for purposes of this invention is naratriptan hydrochloride.

The dosage form preferably comprises from about 0.5 mg. to about 5.0 mg. of naratriptan (based on the weight of the base, in whatever form the naratriptan is present), and more preferably comprises from about 1.0 mg. to about 2.5 mg. of naratriptan, or about 1.0 mg. or about 2.5 mg. specifically, of naratriptan (corresponding to 1.11 or 2.78 mg. of naratriptan hydrochloride). In addition, the dosage form preferably yields a tₘₐₓ for the naratriptan of from about 1.5 to about 4.5 hours, preferably from about 2.0 to about 4.0 hours, whether determined during a migraine-free period or during an attack.

Almotriptan malate is chemically designated as 1-[[[3-[2-(dimethylamino)ethyl]-1H-indol-5-yl]methyl]sulfonyl]pyrrolidine (+)-hydroxybutanedioate (1:1), and has the following chemical structure when present as the malate:

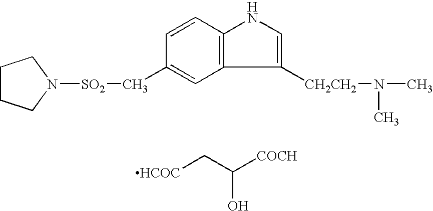

For purposes of this invention, almotriptan can be administered as the base or as any pharmaceutically acceptable salt that demonstrates adequate stability upon storage and bioavailability upon administration, but a preferred form of almotriptan for purposes of this invention is almotriptan malate.

The dosage form preferably comprises from about 2.5 mg. to about 15.0 mg. of almotriptan (based on the weight of the base, in whatever form the almotriptan is present), and more preferably comprises from about 6.25 mg. to about 12.5 mg. of almotriptan, or about 6.25 mg. or about 12.5 mg. specifically, of almotriptan. In addition, the dosage form preferably yields at for the almotriptan of from about 0.5 to about 4.0 hours, preferably from about 1.0 to about 3.0 hours, whether determined during a migraine-free period or during an attack.

Frovatriptan is preferably administered as the succinic acid salt, in an amount of from about 1.0 to about 5.0 mg., preferably about 2.5 mg (based on the weight of frovatriptan). In addition, the dosage form preferably yields a tₘₐₓ for the frovatriptan of from about 0.5 to about 4.0 hours, whether determined during a migraine-free period or during an attack.

The combined dosage forms of the present invention can be evaluated by one or more of the following clinical endpoints in patients suffering from moderate to severe headache. In one embodiment, the combined dosage form is not inferior when compared to each of the active ingredients administered individually against one or more or all of the following endpoints:
Percentage of patients pain free at two hours;
Mean reduction in pain intensity at 60, 120, 180 and 240 minutes after administration (as measured on 100 mm VAS);
Percentage of patients that remain pain free without rescue medication after 8 and/or 24 hours;
Percentage of patients with headache response within two hours of treatment (i.e. pain reduction from moderate or severe to mild or none);
Percentage of patients with sustained headache response without rescue medication after 8 and/or 24 hours;
Reduction in nausea at 2, 4 or 8 hours;
Reduction in photophobia at 2, 4 or 8 hours; and
Reduction in phonophobia at 2, 4, or 8 hours;

In an even more preferred embodiment, the combination dosage form is superior to each of the active ingredients administered individually when measured against one or more of the foregoing endpoints, and not inferior when measured against one or more or the remainder of the endpoints. Thus, for example, the combination dosage form is preferably superior to each of the active ingredients administered individually when measured against one or more of the following endpoints, and not inferior when measured against one or more or the remainder of the following endpoints:
Mean reduction in pain intensity at 2 hours after administration (as measured on 100 mm VAS);
Percentage of patients with headache response at 2 hours after treatment (i.e. pain reduction from moderate or severe to mild or none);
Reduction in nausea at 2 hours after administration;
Reduction in photophobia at 2 hours after administration; and
Reduction in phonophobia at 2 hours after administration;

Alternatively, the methods can be practiced as a stand-alone treatment against any of the foregoing symptoms. Thus, for example, the invention may be characterized as a method of treating photophobia and phonophobia using the dosage forms of the present invention, or for preventing rebound headache within 24 hours of administration.

The dosage forms of the present invention can take various forms, including oral solutions, oral suspensions, powders for oral suspension, tablets, capsules, mucoadhesive films, and orally dissolving tablets, among others. The active ingredients can be in one unitary formulation, so that each is released at the same rate when dissolved in the stomach. The unitary formulation can be a conventional immediate release formulation, or a fast release formulation. For purposes of this disclosure, the term “fast release” is defined to mean that the dosage form yields a dissolution or disintegration time of less than about 30, 20, 15, 10, 5 or 3 minutes when tested according to USP 28 <701> or USP 28 <711> (Q=85%). The term “immediate release” is defined to mean that the dosage form yields a dissolution or disintegration time of less than about 90, 60 or 45 minutes (preferably 60 minutes), and typically greater than about 5, 10, 15 or 20 minutes preferably 20 minutes), when tested according to USP 28 <701> or USP 28 <711> (Q=85%).

Of course, it is also possible, and in most instances preferable, to formulate the active ingredients in two separate vehicles, so that differing pharmacokinetic profiles are observed for the diclofenac and the serotonin agonist. This can be done, for example, in the form of a bilayer tablet, that contains a “fast release” layer containing the diclofenac, and an “immediate release” layer that contains the serotonin agonist (measured as defined above for the terms “fast release” and “immediate release.” The layers could be compressed one against the other, so that each is exposed immediately to biological fluids upon ingestion, or one could form the outer layer of a shell that must dissolve completely before exposing the inner/second layer to the biological fluids. Alternatively, separate beads that have different release profiles could be constructed for containing the diclofenac and the serotonin agonist, and proportionate amounts of the beads could be added to a hard gelatin capsule in the preparation of a capsule dosage form.

Therefore, in one embodiment the diclofenac potassium and serotonin agonist achieve fast release when tested according to USP 28 <701> or USP 28 <711> (Q=85%). In another embodiment the diclofenac potassium and serotonin agonist achieve immediate release when tested according to USP 28 <701> or USP 28 <711> (Q=85%). In yet another embodiment the diclofenac potassium achieves fast release, and the serotonin agonist achieves immediate release, when tested according to USP 28 <701> or USP 28 <711> (Q=85%).

The invention also contemplates the concomitant administration of a short acting NSAID and a 5-HT_{1B/1D} receptor agonist in separate dosage forms. These separate dosage forms preferably employ the same dose amounts, and the same pharmacokinetics, as described above for each ingredient in a combination dosage form. Likewise, when combined in a concomitant administration regime, the separate dosage forms preferably meet the clinical endpoints described above. The invention further contemplates kits that comprise the separate dosage forms in a unitary package, with appropriate instructions for use.

For purposes of this invention, the term “concomitant administration” shall refer to “simultaneous administration” or “co-timely administration.” The term “co-timely” as to drug administration shall mean administration of a second drug for migraine relief while a first drug for migraine relief is present in a therapeutically effective amount. It is to be understood that in some instances this will require sequential administration. In some instances, multiple routes of administration will be employed such as intravenous or subcutaneous injection of an 5-HT_{1B/1D} agonist, while a short acting NSAID is taken orally from prior to or subsequent to such 5-HT_{1B/1D} agonist injection.

Buffering agents are not critical to the invention, but are preferably used to provide a rapid rate of onset for the diclofenac. In a preferred embodiment, the buffering agent controls the pH of the portion of the formulation that contains diclofenac when dissolved in water, and preferably yields a pH greater than about 6.8, 7.0, 7.2, or 7.4, and less than about 7.8, 7.7 or 7.6, when mixed with 50 ml or 100 or 200 ml. of water at 25 degrees Celsius. Particularly preferred buffering agents are alkali metal carbonates and bicarbonates and these agents are preferably employed in a weight ratio relative to the diclofenac of greater than about 1:5, 2:5, 2:1, 3:1 or 5:1. If desired, an upper limit on the buffer:diclofenac ratio can be placed at about 20:1, 10:1, 5:1, 1:1, 4:5 or 3:5. Ranges can be selected from any two of the foregoing values that are mathematically possible. In a preferred embodiment, the buffer:diclofenac weight ratio ranges from about 1:5 to about 4:5. Particularly preferred alkali metal bicarbonates are sodium bicarbonate and potassium bicarbonate.

### EXAMPLES

### Example 1

### Formulations

A suitable dose of a 5-HT_{1B/1D} Agonist or other migraine agent can be combined in a therapeutically effective amount (TE) with diclofenac potassium to arrive at the following formulations:

Composition dissolving instantly in water

| Active ingredients | | |
|---|---|---|
| 1) Diclofenac potassium salt*: | 50 | mg |
| 2) 5-HT_{1B/1D} Agonist or other migraine agent | TE | |
| 3) Potassium bicarbonate: | 22 | mg |
| 4) Mint flavoring on maltodextrin (1:2000)**: | 60 | mg |
| 5) Aniseed flavoring on maltodextrin (1:1000)***: | 104 | mg |
| Excipients and adjuvants | | |
| 6) Saccharin: | 4 | mg |
| 7) Aspartame: | 10 | mg |
| 8) Mannitol: | 50 | mg |
| 9) Saccharose****q.s.: | 2 | g |

*If it is desired to prepare compositions based on diclofenac sodium salt, it is advantageous to use sodium bicarbonate in a quantity of approximately 38% by weight based on the weight of the diclofenac sodium salt present.
Sodium carbonate may also be added to the sodium bicarbonate, maintaining the following optimum proportions: 27% of sodium bicarbonate and 4-5% of sodium carbonate, always based on the amount by weight of diclofenac sodium salt present.
**The title of the pure mint essence, as obtained according to the Dean-Stark method, is of 18% by weight; the related amount is therefore in this case of 10.8 mg.
***The title of the pure anise essence, as obtained according to the Dean-Stark method, is of 14.5% by weight, the related amount is therefore in this case of 16 mg.
****The presence of saccharose is not strictly necessary; in its absence, a composition having a very limited granulate content is obtained which is perfectly 20 soluble in contact with water. In that case, nothing is changed from the point of view of tolerability in contact with the mucosa and. from the point of view of the palatability of the drinkable solution.

### Preparation

Components 1, 2, 3, 6, 7 and 8 are mixed in a suitable mixer, and the mixture so obtained is wetted with 95% ethanol. Granulation is carried out with a 66 mm mesh and the granulate is preferably dried in current of air.

Components 4, 5 and 9, which have already been granulated using a mesh of the same granulometry, are then added and the whole is mixed. The mixture is then introduced into a metering machine for filling packets or similar containers.

Tablet for dissolving in the mouth

| Active ingredients | | |
|---|---|---|
| 1) Diclofenac potassium salt*: | 50 | mg |
| 2) 5-HT_{1B/1D} Agonist or other migraine agent | TE | |
| 3) Potassium bicarbonate: | 35 | mg |
| 4) Mint flavoring on maltodextrin** | 50 | mg |
| (1:2000) + gum arabic (E 414): | | |
| 5) Aniseed flavoring (1:1000) | 120 | mg |
| on maltodextrin*** + silicon | | |
| dioxide(E551): | | |
| Excipients and adjuvants | | |
| 6) Saccharin: | 50 | mg |
| 7) Aspartame: | 12 | mg |
| 8) Mannitol: | 20 | mg |
| 9) Saccharose****: | 300 | mg |

* to ****see Example 1

Two layered tablet (fast and slow release)
Fast release layer

| | | |
|---|---|---|
| 1) Diclofenac potassium salt: | 15 | mg |
| 2) Potassium bicarbonate: | 30 | mg |
| 3) Lactose: | 13.2 | mg |
| 4) Maize starch (intragranular): | 6 | mg |
| 5) Methyl cellulose: | 0.12 | mg |
| 6) Sodium laurylsulfate: | 0.06 | mg |
| 7) Maize starch (extragranular): | 9 | mg |
| 8) Crospovidone: | 0.6 | mg |
| 9) Sodium carboxymethylstarch: | 1.5 | mg |
| 10) Magnesium stearate: | 2.7 | mg |
| 11) Colloidal silicon dioxide: | 0.6 | mg |

Slow release layer

| 1) 5-HT_{1B/1D} Agonist or other migraine agent | TE | |
|---|---|---|
| 2) Lactose: | 32.2 | mg |
| 3) Polyvinylpyrrolidone: | 1.16 | mg |
| 4) Hydroxypropylmethylcellulose: | 70 | mg |
| 5) Magnesium stearate: | 0.84 | mg |
| 6) Colloidal silicon dioxide: | 0.21 | mg |
| 7) Talc: | 3.92 | mg |
| 9) Polyethylene glycol: | 0.56 | mg |

Tablet

| 1) Diclofenac potassium salt: | 50 | mg |
|---|---|---|
| 2) 5-HT_{1B/1D} Agonist or other migraine agent | TE | |
| 3) Mannitol: | 50 | mg |
| 4) Potassium bicarbonate: | 22 | mg |
| 5) Maize starch (intragranular): | 10 | mg |
| 6) Methyl cellulose: | 0.2 | mg |
| 7) Sodium laurylsulfate: | 0.1 | mg |
| 8) Maize starch (extragranular): | 15 | mg |
| 9) Crospovidone: | 1.0 | mg |
| 10) Sodium carboxymethylstarch: | 2.5 | mg |
| 11) Magnesium stearate: | 4.5 | mg |
| 12) Colloidal silicon dioxide: | 10 | mg |

### Example 2

### Comparative Efficacy of Diclofenac Powder Against Migraine Headache

A randomized, double-blind, double-dummy multi-center, single dose, placebo- and active-controlled crossover study, with an eight hour evaluation was undertaken in adult migraine patients. 328 migraine patients with or without aura according to HIS criteria were randomized among treatments and a comparison made among treatments with a 50 mg. diclofenac potassium sachet formulation prepared substantially as described in Example 1, and demonstrating a tₘₐₓ of about 14 minutes, a 50 mg. diclofenac potassium sugar coated tablet marketed commercially as Cataflam®, and demonstrating a tₘₐₓ of about 52 minutes, and placebo. Patients were randomized to treatment for three separate migraine attacks, each attack treated with a different study medication. Results are reported in Table 1.

TABLE 1
Pain on Verbal Scale

| | Parameter | | |
|---|---|---|---|
| | Diclofenac-K | Diclofenac-K | |
| | Sachet | Tablet | Placebo |
| | % of patients | % of patients | % of patients |
| Pain free at 2 hours | | | |
| ITT pop | 24.7% | 18.5% | 11.7% |
| PP pop | 23.6% | 17.8% | 12.9% |
| Mod-sev | 24.2% | 17.0% | 12.5% |
| baseline pain | | | |
| Headache response 2 | 46.0% | 41.6% | 24.1% |
| hours* | | | |
| Sustained response | 36.8% | 30.9% | 18.4% |
| Sustained pain free** | 22.3% | 15.1% | 9.4% |

*pain reduction from moderate or severe to mild or none
**no recurrence of pain and no rescue within 24 hours

### Example 3

### Efficacy of Diclofenac Powder Against Primary and Secondary Migraine Endpoints

A phase III clinical trial was undertaken in adult migraine patients. 690 migraine patients were randomized among treatments and a comparison made among treatments with a 50 mg. diclofenac potassium sachet formulation prepared substantially as described in Example 1, and demonstrating a tₘₐₓ of about 14 minutes, and placebo. The efficacy of the treatment against four primary endpoints (headache pain, nausea, photophobia and phonophobia) are reported in Table 2.

TABLE 2

| | PRO-513 | Placebo |
|---|---|---|
| Headache Pain^{a} | | |
| Number of Subjects | 343 | 347 |
| No Pain | 86 (25.1%) | 35 (10.1%) |
| Mild, Moderate, or Severe Pain | 257 (74.9%) | 312 (89.9%) |

| | |
|---|---|
| P-Value^{b} | <0.001 |

| | | |
|---|---|---|
| Nausea^{a} | | |
| Number of Subjects | 343 | 347 |
| No Nausea | 222 (64.7%) | 183 (52.7%) |
| Mild, Moderate, or Severe Nausea | 121 (35.3%) | 164 (47.3%) |

| | |
|---|---|
| P-Value^{b} | 0.002 |

| | | |
|---|---|---|
| Photophobia^{a} | | |
| Number of Subjects | 343 | 347 |
| No Photophobia | 139 (40.5%) | 95 (27.4%) |
| Mild, Mod., or Sev. Photophobia | 204 (59.5%) | 252 (72.6%) |

| | |
|---|---|
| P-Value^{b} | <0.001 |

| | | |
|---|---|---|
| Phonophobia^{a} | | |
| Number of Subjects | 343 | 347 |
| No Photophobia | 152 (44.3%) | 95 (27.4%) |
| Mild, Mod., or Sev. Photophobia | 191 (55.7%) | 252 (72.6%) |

| | |
|---|---|
| P-Value^{b} | <0.001 |

^{a}Based on Assessments at 2 Hours Post-Dose
^{b}P-Value from a Cochran-Mantel-Haenszel test, stratified by analysis center.

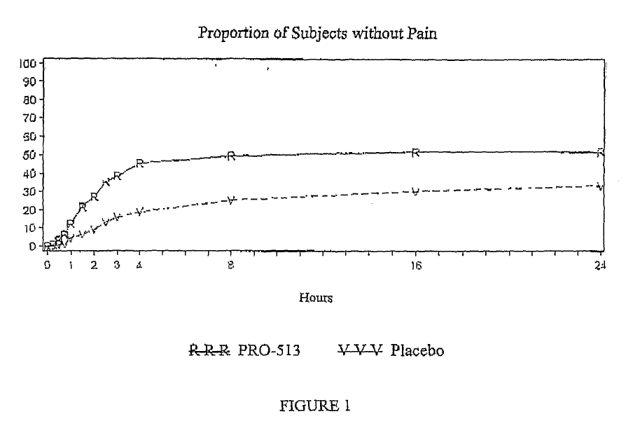

A graphical summary of headache intensities during the first twenty four hours after treatment, comparing a fast release diclofenac sachet formulation and placebo, is reported in FIG. 1. Sustained pain free and recurrence rates are reported below in Table 3:

TABLE 3
Sustained Pain Free Response Rates

| Sustained PF | PRO-513 (N = 343) | Placebo (N = 347) | P-Value^{a} |
|---|---|---|---|
| Yes | 65 (19.0%) | 25 (7.2%) | <0.001 |
| No | 278 (81.0%) | 322 (92.8%) | |

Recurrence Rates

| Recurrence | PRO-513 (N = 86) | Placebo (N = 35) |
|---|---|---|
| Yes | 21 (24.4%) | 10 (28.6%) |
| No | 65 (75.6%) | 25 (71.4%) |

Time to Recurrence

| | Median Time (Hours) | 95% C.I. |
|---|---|---|
| PRO-513 | >24 | (24.0 to >24) |
| Placebo | >24 | (24.0 to >24) |

^{a}P-Value from a Cochran-Mantel-Haenszel test, stratified by analysis center.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of treating photophobia and phonophobia comprising administering a dosage form that comprises:
a) a therapeutically effective amount of diclofenac, or a pharmaceutically acceptable salt thereof, and
b) a therapeutically effective amount of a 5-HT_{1B/1D} receptor agonist, or a pharmaceutically acceptable salt or ester thereof.

2) (canceled)

3) The method of claim 1 wherein said fixed dosage form comprises a fast release portion comprising said diclofenac potassium, and said diclofenac potassium has a normalized Cₘₐₓ of from about 0.026 ml⁻¹ to about 0.05 ml⁻¹.

4) The method of claim 1 wherein said fixed dosage form comprises a fast release portion comprising said diclofenac potassium, and said diclofenac potassium has a tₘₐₓ of less than about 30 minutes.

5) The method of claim 1 wherein said dosage form comprises from about 25 to about 100 mg. of sumatriptan as sumatriptan succinate, and from about 15 to about 75 mg. of diclofenac potassium.

6) The method of claim 1 wherein said dosage form comprises from about 10 to about 60 mg. of eletriptan as eletriptan hydrobromide, or from about 2.5 mg. to about 15 mg. of rizatriptan as rizatriptan benzoate, or from about 1.5 mg. to about 7.5 mg. of zolmitriptan, or from about 0.5 mg. to about 5.0 mg. of naratriptan as naratriptan hydrochloride, or from about 2.5 mg. to about 15.0 mg. of almotriptan as almotriptan malate, or from about 1.0 to about 5.0 mg. of frovatriptan and frovatriptan succinate, and from about 15 to about 75 mg. of diclofenac potassium.

7) (canceled)

8) (canceled)

9) (canceled)

10) (canceled)

11) (canceled)

12) (canceled)

13) (canceled)

14) (canceled)

15) A method of treating migraine comprising concomitantly orally administering a dosage form that comprises:
a) a therapeutically effective amount of diclofenac, or a pharmaceutically acceptable salt thereof, and
b) a therapeutically effective amount of a 5-HT_{1B/1D} receptor agonist, or a pharmaceutically acceptable salt or ester thereof.

16) (canceled)

17) (canceled)

18) (canceled)

19) (canceled)

20) (canceled)

21) (canceled)

22) (canceled)

23) (canceled)

24) (canceled)

25) (canceled)

26) (canceled)

27) The method of claim 15 wherein said dosage form reduces the intensity of pain more than either of the 5-HT_{1B/1D} receptor agonist or diclofenac potassium components alone, at eight hours and twenty-four hours post-administration.

28) The method of claim 15 wherein the dosage form reduces the eight hour recurrence rate of migraine more than either of the 5-HT_{1B/1D} receptor agonist or diclofenac potassium components alone.

29) The method of claim 15 wherein the dosage form reduces the twenty-four hour recurrence rate of migraine more than either of the 5-HT_{1B/1D} receptor agonist or diclofenac potassium components alone.

30) The method of claim 15 wherein the dosage form reduces nausea more than either of the 5-HT_{1B/1D} receptor agonist or diclofenac potassium components alone.

31) (canceled)

32) (canceled)

33) (canceled)

34) A fixed combination dosage form comprising diclofenac or a pharmaceutically acceptable salt thereof and a 5-HT_{1B/1D} receptor agonist.

35) The dosage form of claim 34 wherein said fixed dosage form comprises a fast release portion comprising diclofenac potassium, and said diclofenac potassium has a normalized Cₘₐₓ of from about 026 ml⁻¹ to about 0.05 ml⁻¹.

36) The dosage form of claim 34 wherein said fixed dosage form comprises a fast release portion comprising diclofenac potassium, and said diclofenac potassium has a tₘₐₓ of less than about 30 minutes.

37) The dosage form of claim 34 wherein said fixed dosage form comprises a fast release portion comprising diclofenac potassium, and an immediate release portion comprising said 5-HT_{1B/1D} receptor agonist.

38) The dosage form of claim 34 wherein said fixed dosage form is an immediate release dosage form.

39) The dosage form of claim 34 wherein said fixed dosage form is a fast release dosage form.

40) The method of claim 34 wherein said dosage form comprises from about 25 to about 100 mg. of sumatriptan as sumatriptan succinate, and from about 15 to about 75 mg. of diclofenac potassium.

41) The method of claim 34 wherein said dosage form comprises from about 10 to about 60 mg. of eletriptan as eletriptan hydrobromide, or from about 2.5 mg. to about 15 mg. of rizatriptan as rizatriptan benzoate, or from about 1.5 mg. to about 7.5 mg. of zolmitriptan, or from about 0.5 mg. to about 5.0 mg. of naratriptan as naratriptan hydrochloride, or from about 2.5 mg. to about 15.0 mg. of almotriptan as almotriptan malate, or from about 1.0 to about 5.0 mg. of frovatriptan and frovatriptan succinate, and from about 15 to about 75 mg. of diclofenac potassium.

42) (canceled)

43) (canceled)

44) (canceled)

45) (canceled)

46) (canceled)

47) The dosage form of claim 34 wherein said dosage form is a powder for oral suspension.

48) The dosage form of claim 34 wherein said dosage form is a tablet or capsule

49) The dosage form of claim 34 wherein said dosage form is a liquid solution or suspension.

50) A fixed combination dosage form comprising:
a) diclofenac or a pharmaceutically acceptable salt thereof, wherein greater than 85% of said diclofenac or diclofenac salt is released from said formulation in less than twenty minutes when tested in water having a pH of 6.8 at 37° C. at 50 RPM using a USP type II dissolution apparatus; and
b) a 5-HT_{1B/1D} receptor agonist, wherein greater than 85% of said 5-HT_{1B/1D} receptor agonist is released from said formulation in less than sixty minutes when tested in water buffered by 0.1M HCl at 37° C. at 50 RPM using a USP Type II dissolution apparatus.
